(19) United States
(12) Patent Application Publication
(10) Pub. No.: US 2008/0293671 A1
(43) Pub. Date: Nov. 27, 2008

(54) Combination of Polychitosamine and Fibrate for the Prevention and Treatment of Hyperlipidemia

(75) Inventors: Andre Aube, St-Sauveur (CA); Ryszard Brzezinski, Sherbrooke (CA); Gilles Dupuis, Sherbrooke (CA); Jean-Guy Lehoux, Sherbrooke (CA)

Correspondence Address: CHOATE, HALL & STEWART LLP, TWO INTERNATIONAL PLACE, BOSTON, MA, 02110, US

(73) Assignee: DNP Canada, Inc., Montreal, QC (CA)

(21) Appl. No.: 12/088,387

(22) PCT Filed: Sep. 28, 2006
(86) PCT No.: PCT/CA2006/001603

§ 371 Date: Mar. 27, 2008

Related U.S. Application Data
(60) Provisional application No. 60/721,102, filed on Sep. 28, 2005.

(51) Int. Cl. A61K 31/722; A61P 3/06

(52) U.S. Cl. 514/55

(57) ABSTRACT

The present invention relates to pharmaceutical compositions comprising a fibrate (e.g. ciprofibrate, gemfibrozil, benzafibrate, and fenofibrate) and a polychitosamine (e.g. chitosan) having molecular weights of less than 650 Kda and degrees of deacetylation of from 70% to 100%. Uses of said compositions for the treatment of hyperlipidemia and associated conditions (e.g. hypercholesterolemia, atherosclerosis, coronary heart disease and cardiovascular disease) or as functional foods are also disclosed.

### FIELD OF THE INVENTION

The present invention relates to the field of therapeutic agents useful in lowering cholesterol or improving the ratio of HLD:LDL (particularly lowering low-density lipoproteins and/or increasing high density lipoproteins) and/or cholesteryl esters, triglycerides, phospholipids and fatty acids in a mammal, such as a human. More particularly, the invention relates to combination therapies, uses, and pharmaceutical compositions having greater therapeutic benefits than monotherapies using the same therapeutic substances.

### BACKGROUND OF THE INVENTION

It is well known that hyperlipidemic conditions associated with elevated concentrations of total cholesterol and low-density lipoprotein (LDL) cholesterol are major risk factors for cardiovascular disease such as atherosclerosis. Numerous studies have demonstrated that a low plasma concentration of high density lipoprotein (HDL) cholesterol (good cholesterol) is a powerful risk factor for the development of atherosclerosis (Barter and Rye. Atherosclerosis, 121, 1-12 (1996)). HDL is one of the major classes of lipoproteins that function in the transport of lipids through the blood. The major lipids found associated with HDL include cholesterol, cholesteryl ester, triglycerides, phospholipids and fatty acids. The other classes of lipoproteins found in the blood are low density lipoprotein (LDL), intermediate density lipoprotein (IDL), and very low density lipoprotein (VLDL). Since low levels of HDL cholesterol increase the risk of atherosclerosis, methods for elevating plasma HDL cholesterol would be therapeutically beneficial for the treatment of cardiovascular diseases such as atherosclerosis. Cardiovascular diseases include, but are note limited to, coronary heart disease, peripheral vascular disease, and stroke.

An approach for addressing hyperlipidemia has been the use of polychitosamine, a substance derived from the shell of crustaceans. Polychitosamine is a highly deacetylated and low molecular weight derivative of chitin. As described in Canadian patent no. 2,085,292, enzymatic treatment of chitosan with a hydrolase enzyme results in chitosan derivatives having a decreased molecular weight, decreased viscosity and increased solubility of chitosan in water.

In its mechanism of action, polychitosamine contains free amine groups which attach themselves to lipids such as cholesterol via ionic bonds while in the intestinal tactus, forming an indissociable complex that is eventually excreted. Polychitosamine therefore prevents lipids such as cholesterol from ever entering the bloodstream and adding to the total cholesterol content. It is commonly used in dosages of 400-800 mg/three times a day, with meals and is indicated towards the obtention of a healthy lipid profile and more particularly a healthy cholesterol profile. Polychitosamine is well tolerated by patients. Reported side-effects at the dosage levels indicated above have been mild to none. (Veneroni G, Veneroni F, Contos S et al. Effect of a new chitosan dietary integrator and hypocaloric diet on hyperlipidemia and overweight in obese patients. Acta Toxicol the 1996 17(1):53-60).

Polychitosamine is currently marketed by Magistral Biotech under the brand names Libracol® (HEP-30) in 800 mg gelatine capsules.

Another known therapy is the use of fibrate for lowering blood cholesterol. Fibrates are cholesterol-lowering drugs that are primarily effective in lowering triglycerides and, to a lesser extent, in increasing HDL-cholesterol levels. Several fibrate drugs are commercially available such as Gemfibrozil available under the brand name LOPID. LOPID is the most widely used fibrate in the United States and can be very effective for patients with triglyceride levels. However, it is not very effective for lowering the LDL-cholesterol. As a result, it is used less often than other drugs and patients with heart disease for whom LDL-cholesterol lowering is the main goal of treatment. It should also be noted that Gemfibrozil as a monotherapy is not recommended by the United States Food and Drug Administration for patients with heart disease.

On the other hand, fibrates are generally well-tolerated by most patients. Gastrointestinal complaints are the most common side effects and fibrates appear to increase the likelihood of developing cholesterol gallstones. Fibrate can increase the effective medications that thin the blood. Fibrates are usually given in two daily doses, 30 minutes before the morning and evening meals. The reductions in triglycerides generally are in the range of 20% to 50% with increase in HDL cholesterol of 10% to 15%. Other fibrate drugs include Stiprofibrate; Belzafibrate; and Finofibrate.

Another well-known therapeutic approach for treating or preventing hyperlipidemic conditions is the use of HMG-CoA reductase inhibitors (including class of therapeutics commonly called “statins”). Statins are known for reducing blood serum levels of LDL cholesterol by competitive inhibition of the biosynthetic step. Several statins have been developed and commercialized throughout the world. Atorvastatin calcium sold in North America under the brand Lipitor® is a potent reductase Inhibitor. However, warning side effects from use of statins include liver dysfunction, skeletal muscle myopathy, rhabdomyolysis and acute renal failure. Some of these effects are exacerbated when statins are taken in greater doses. For example, a patient treated with 10 mg per day of Lipitor may notice mild side effects whereas the side effects may greatly increase by simply raising the daily dose to 20 mg per day. In fact, on Aug. 8^{th}, 2001, the maker of cerivastatin voluntarily pulled the medicine off the market because of numerous deaths associated with its use. The deaths were caused by complications of severe muscle breakdown.

A number of combination therapies for the treatment of cardiovascular disease, which include statin as one of the active components, are known. More precisely, the combination therapy of statin and fibrate is well-known. However, recent studies have shown that the risk of developing rhabdomyolysis increases 1400 times when statins are combined with fibrates. This risk is even more prevalent in patients with diabetes who combine these two types of medications, producing as many as 1 in 500 people affected with rhabdomyolysis (http://www.wnbc.com/drmaxgomez/393553/details.html).

Another example of a combination therapy of fluvastatin and niceritrol is described by J. Sasaki et al. (Int. J. Clin. Pharmacol. Ther., July; 33(7), 420-6 (1995)), Those researchers conclude that the combination of fluvastatin with niceritrol “a dose of 760 mg/day does not appear to augment or attenuate beneficial effects of fluvastatin.”

L. Cashin-Hemphill et al. (J. Am. Med. Assoc., 264 (23), 3013-17 (1990)) describe beneficial effects of a combination therapy of colestipol and niacin on coronary atherosclerosis.

A combination therapy of acipimox and simvastatin shows beneficial effects in patients having high triglyceride levels (N. Hoogerbrugge et al., J. Internal Meds, 241, 151-55 (1997)).

In a study on the reduction of inflammatory biomarkers by statin, fibrate and combination therapy among diabetic patients with mixed dyslipidemia (G Am Coll Cardiol. 2006 Jul. 18; 48(2):396-401), it has been reported that the anti-inflammatory effects in combination therapies was no more effective than either form of monotherapy.

Thus, there is a need for seeking alternative cholesterol lowering strategies that are safe and do not present side effects severe enough to create additional health problems such as rhabdomyolysis. More particularly, there is a need to provide new therapeutic approaches having the advantages afforded by combination therapies (complementary mode of action) all the while being safe and efficient. The present invention provides such new therapeutic approach in the form of new combinations of cholesterol-lowering agents that are compatible, thus safe to use all the while being at least as effective as the corresponding monotherapies for each of the corresponding cholesterol-lowering agents present in the combination.

### SUMMARY OF THE INVENTION

An embodiment of the present invention provides a pharmaceutical composition comprising: a) a pharmaceutically effective amount of fibrate; b) a pharmaceutically effective amount of polychitosamine and optionally c) a pharmaceutically acceptable carrier.

An embodiment of the present invention also provides the use of the above-defined pharmaceutical composition to increase the level of HDL in the blood of a mammal.

A further embodiment of the present invention provided the use of the above-defined pharmaceutical composition to lower the level of low density lipoproteins in the blood of a mammal.

A further embodiment of the present invention provides a method for lowering the concentration of low density lipoprotein in the blood of a mammal and/or improving the ratio of HLD:LDL in such a mammal by increasing the level of high density lipoproteins; the method comprising the step of administering a pharmaceutical composition of the invention or a) a first amount of a fibrate and b) a second amount of polychitosamine; wherein the first and second amounts together comprise a therapeutically effective amount.

Another embodiment provides a method for the prophylaxis or treatment of hyperlipidemia or hyperlipidemia-associated condition comprising administering to a patient: a) a first amount of a fibrate and b) a second amount of polychitosamine; wherein the first and second amounts together comprise a therapeutically effective amount.

Further embodiment provides a kit for the prophylaxis or treatment of hyperlipidemia or hyperlipidemia-associated condition in a mammal comprising a plurality of daily doses of dosage forms of a fibrate, a plurality of daily doses of dosage forms of polychitosamine together, and treatment or regimen written instructions.

The present invention has the advantage of providing an alternative therapy for lowering blood cholesterol by unexpectedly combining cholesterol-lowering active agents that are compatible in nature. Such combination allows the use of a reduced amount of each of the cholesterol-lowering active agents, thereby reducing side effects.

### DETAILED DESCRIPTION OF THE INVENTION

The present invention provides combination therapies which have a dual cholesterol-lowering mechanism that allows effective cholesterol lowering with reduced side effects as compared to the corresponding monotherapies. When referring to dual cholesterol-lowering mechanism, it is meant, lowering of total blood cholesterol through two distinct physiological modes of action.

The present invention improves efforts for preventing and/or treating hyperlipidemia, such as by reducing serum cholesterol, by providing a combination therapy approach and a novel pharmaceutical composition that provides therapeutic benefits such as safety and reduced side effects not available through other known mono or multiple therapies for hyperlipidemia. The present invention provides benefits afforded by fibrate in a combination therapy that would be otherwise contraindicated if used in combination with other known cholesterol-lowering agents such as statin. The polychitosamine/fibrate combination of the present invention allows a patient to benefit from the complementary dual cholesterol-lowering effects of each of these agents without adverse effects caused by incompatibility of the two agents. The invention thus lies in the new combination of polychitosamine and fibrate as a safe and efficient therapy for lowering blood cholesterol.

The present invention provides a combination therapy for lowering blood cholesterol which comprises a combination of at least one fibrate and one polychitosamine. Preferably, the fibrate is selected from the group consisting of ciprofibrate, gemfibrozil; benzafibrate; and fenofibrate. Also preferably, the polychitosamine is a chitosan salt having a molecular weight of less than about 650 kDa and has a deacetylation degree of about 70% to 100%.

The present invention also provides a method for lowering blood cholesterol and/or increasing the HDL/LDL ratio by increasing the level of HDL by administering to a mammal the pharmaceutical composition of the present invention or by administering a composition comprising an effective amount of fibrate and polychitosamine.

The present invention also provides a method for the prophylaxis or treatment of hyperlipidemia or hyperlipidemia associated condition comprising administering to a patient the pharmaceutical composition of the present invention or a composition comprising an effective amount of at least a fibrate or polychitosamine.

The present invention further provides an oral dosage form of a pharmaceutical composition comprising a combination of polychitosamine and fibrate wherein the dosage form provides to a mammal in need of treatment by the polychitosamine and fibrate, a therapeutically effective dose of polychitosamine and therapeutically effective quantity of fibrate into the blood of said mammal.

The present invention also provides a kit for the prophylaxis or treatment of hyperlipidemia in a mammal the kit comprising a plurality of daily doses of dosage forms of a fibrate and a polychitosamine together with a treatment regiment instructions.

The combinations of the present invention will have a number of advantages. For example, through dosage adjustment and medical monitoring; the individual dosages of the therapeutic compounds used in the combinations of the present invention will be lower than the typical dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Another advantage provided by the combination therapy of the present invention is that it provides a dual cholesterol-lowering mechanism that consists of complementary effects or complementary modes of action for lowering blood cholesterol. For example, fibrate can control blood serum cholesterol levels by decreasing serum triglycerides via reduced hepatic production and increased triglyceride clearance by the peripheral tissues and increase HDL. In contrast, polychitosamine can block the migration of cholesterol and/or other lipids such as cholesteryl esters and triglycerides from the intestinal tractus to the blood stream, and the reabsorption of bile acids from the intestinal tractus to the blood stream.

A further advantage of the combination therapy provided by the present invention is that it is safe to use due to the very low incidence of side effects or adverse events caused by the combination of same. In the present case, the fibrate is combined with a polychitosamine which is a natural occurring biopolymer which is also known to be useful in lowering blood cholesterol. As previously indicated, polychitosamine is currently marketed by Magistral as a dietary supplement under the brand Libracol® (HEP 30) in 800 ml gelatin capsules.

### DEFINITIONS

As used herein the term “fibrate” refers to a class of amphipathic carboxylic acids. They are used for a range of metabolic disorders mainly hypercholesterolemia (high cholesterol), hyperlipidemia and are therefore hyperlipidemic agents.

As used herein, “chitin” refers to a polymer formed primarily of repeating units of β (14) 2-acetamido-2-deoxy-D-glucose (or N-acetylglucasamine), Not every unit of naturally-occurring chitin is acetylated, with about 16% deacetylation.

As used herein, “chitosan” refers to chitin that has been partially or fully deacetylated. Chitosan is a polysaccharide formed primarily of repeating units of β (14) 2-amino-2-deoxy-D-glucose (or D-glucosamine). Further deacetylation of chitin can be achieved by processing of chitin, Deacetylation values can vary with chitin sources and with processing methods.

As used herein the term “polychitosamine” or the term “chitodextrine”, refers to a chitosan polymer having a molecular weight of less than about 650 kDa, preferably about 2-500 kDa, more preferably about 15-200 kDa, still more preferably about 20-100 kDa, yet more preferably about 25-60 kDa, and ideally about 30-60 kDa. In one embodiment, the molecular weight of the polychitosamine is about 30 kDa and in another embodiment, the molecular weight is about 30, 31, 32, 33, 34, 35, 36, 37, 38, 39, 40, 41, 42, 43, 44, 45, 46, 47, 48, 49, or 50 kDa. The polychitosamine can be obtained by cleaving a heavier molecular chain of chitosan. Preferably, polychitosamine 16 highly deacetylated, such as over about 80%, and more preferably over about 89%. Polychitosamine is understood herein to also encompass a chitosan salt formed from any chitosan molecule associated with a negatively charged anion. A series of anions has been used for that purpose. For example, anions derived from inorganic acids such as sulphuric acid (sulphate), phosphoric acid (phosphate), hydrochloric acid (chloride) and organic acids such as malic acid (malate), tartaric acid (tartrate), citric acid (citrate), succinic acid (succinate) and lactic acid (lactate), chitin, chitosan, and their derivatives.

As used herein the term “combination therapy” refers to the administration of two or more therapeutic agents to treat a hyperlipidemic condition, for example atherosclerosis and hypercholesterolemia. Such administration encompasses co-administration of these therapeutic agents in a substantially simultaneous manner, such as in a single capsule having a fixed ratio of active ingredients or in multiple, separate dosage forms for each type of therapeutic agent in a sequential or staggered manner. In either case, the treatment regimen will provide beneficial effects of the drug combination in treating the hyperlipidemic condition.

As used herein, “nutraceuticals” is understood to encompass any ordinary food that has components or ingredients added to give a specific medical or physiological benefit other than a purely nutritional effect. It is also understood to include functional foods, dietary supplements and over the counter products sold without a prescription.

As used herein, ‘functional foods’ is understood to encompass any food consumed as part of a usual diet that is similar in appearance to, or may be, a conventional food, and is demonstrated to have physiological benefits and/or reduce the risk of chronic disease beyond basic nutritional functions.

The phrase “therapeutically effective” is intended to qualify the amount of the active ingredient in the combination therapy that will achieve the goal of reducing or eliminating the hyperlipidemic condition.

The phrase “therapeutic compound” refers to a compound useful in the prophylaxis or treatment of a hyperlipidemic condition, such as, but not limited to, atherosclerosis, hypercholesterolemia, coronary heart disease, and cardiovascular disease.

The phrase <<pharmaceutically effective>> is intended to qualify the amount of the ingredient that will achieve the goal intended by the pharmaceutical composition.

The phrase <<dual cholesterol-lowering mechanism>> means lowering of total blood cholesterol through two distinct physiological modes of action.

### Polychitosamine

Polychitosamine refers to a chitosan polymer having a molecular weight of less than about 650 kDa, preferably about 2-500 kDa, more preferably about 15200 kDa, still more preferably about 20-100 kDa, yet more preferably about 25-60 kDa, and ideally about 30-50 kDa.

Chitosan is a naturally-occurring biopolymer that can also be obtained by partial or complete deacetylation of chitin that is the major component of the exoskeleton of shellfishes and insects. Chitosan is therefore a linear polymer composed of monomers of N-acetyl-2-amino-β-D-glucose and 2-amino-β-D-glucose. The presence of the primary amino groups of the 2-amino-β-D-glucose (D-glucosamine) units confers to chitosan its polycationic (positively charged) character that is neutralized by accompanying negatively charged anions. A series of anions has been used for that purpose. For example, anions derived from inorganic acids such as sulfuric acid (sulfate), phosphoric acid (phosphate), hydrochloric acid (chloride), and a mixture thereof and organic acids such as malic acid (malate), tartaric acid (tartrate), citric acid (citrate), lactic acid (lactate), acetic acid (acetate), formic acid (formate), glycolic acid (glycolate), oxalic acid, succinic acid, ascorbic c, maleic acid, acrylic acid, gluconic acid, glutamic acid, propionic acid and a mixture thereof have been reported as salts of chitosan.

While there exists many extraction methods of the chitin from the crustacean shells, the principles of chitin extraction are relatively simple. In a certain treatment, the proteins are removed in a dilute solution of sodium hydroxide (such as about 1%-10%) at high temperature (such as about 85° C.-100° C.). Shells are then demineralized to remove calcium carbonate. This can be done by treating in a dilute solution of hydrochloric acid (1%-10%) at room temperature. Depending on the seventy of these treatments such as temperature, duration, concentration of the chemicals, concentration and size of the crushed shells, the physico-chemical characteristics of the extracted chitin can vary. For instance, three characteristics of the chitin, such as the degree of polymerization, acetylation, and purity, can be affected. Shell also contains lipids and pigments. Therefore, a decolorizing step is sometimes needed to obtain a white chitin. This can be done by soaking in organic solvents or in a very dilute solution of sodium hypochlorite. Again, these treatments can influence the characteristics of the chitin molecule.

Chitin can be deacetylated partially or totally. Such a deacetylated polymer is called chitosan. Chitosan compounds in a range of up to and exceeding 1×10⁶ molecular weight are derived commercially form chitin. In nature, chitosan is present in cell walls of Zygomycetes, a group of phytopathogenic fungi. Because of its significant content of free amino groups, chitosan has a markedly cationic character and has a positive charge at most pHs. Canadian Patent 2,085,292 discloses the hydrolysis of chitosan, the disclosure of which is incorporated herein by reference. Polychitosamine can be produced by the process described in Canadian Patent 2,085,292, and recovered from solution using the process described in WO 2005/066213-A1 where the chitosan is salted out with a salting-out salt such as but not limited to sulfates, phosphates, citrates, nitrates, malates, tartrates, succinates, propionates, lactates and hydrogen phosphates. More preferably, these salting-out salts may be selected from the group consisting of: ammonium or sodium sulfate; sodium or potassium phosphates; sodium or potassium citrate; sodium tartrate; sodium malate; sodium nitrate; sodium lactate; sodium malonate; sodium succenate; sodium acetate; sodium propionate. Thus, the present invention includes any chitosan derivative obtained by any of the above-mentioned salts.

As an example, the citrate salt of chitosan can be illustrated as follows:

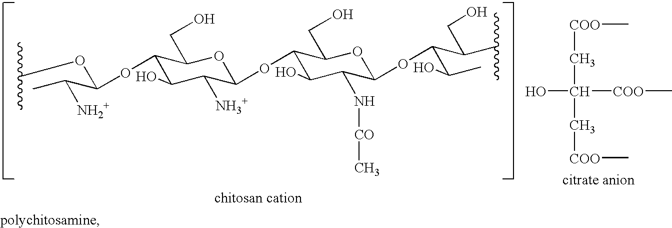

In a mechanism of action, polychitosamine, in particular chitosan, can contain free amine groups which can attach themselves to lipids, such as cholesterol, via ionic bonds while in the intestinal tractus, forming an indissociable complex which is eventually excreted. Polychitosamine therefore can prevent lipids, such as cholesterol, from ever entering the bloodstream and adding to the total cholesterol content. Also, in reaction, the liver eliminates more cholesterol by using biliary acids. Therefore, there is elimination of both food cholesterol and that of biliary acids rich in cholesterol.

### Molecular Weight

Polychitosamine has many potential applications depending on its molecular weight. An average high molecular weight polychitosamine is about 650 kDa. Some applications are typical of medium or low molecular weight polychitosamine, ranging typically about 2-500 kDa. These applications include its use as an antifungal agent; a seed coating for improving crop yield; an elicitor of anti-pathogenic natural reactions in plants; a hypocholesterolemic agent in animals; an accelerator of lactic acid bacteria in breeding; and a moisture-retaining agent for lotions, hair tonics and other cosmetics.

The molecular weight of polychitosamine is a feature that is particular to a certain application. The molecular weight of the native chitin has been reported to be as high as many million Daltons. However, chemical treatment tends to bring down the molecular weight of the polychitosamine ranging from 100 kDa to 150 kDa. Further treatment of the polychitosamine can lower the molecular weight even more. Low molecular weight could be produced by different ways including enzymatic or chemical methods. When the chain becomes shorter, the polychitosamine car be dissolved directly in water without the need of an acid. This is particularly useful for specific applications, such as in cosmetics or in medicine Molecular weight of the polychitosamine can be measured by analytical methods, such as gel permeation chromatography, light scattering, or viscometry. Because of simplicity, viscometry is the most commonly used method.

In one embodiment, the molecular weight of the polychitosamine is about 30 kDa and in another embodiment, the molecular weight is about 30, 31, 32, 33, 34, 35, 36, 37, 38, 39, 40, 41, 42, 43, 44, 45, 46, 47, 48, 49, or 50 kDa. The polychitosamine can be obtained by cleaving a heavier molecular chain of chitosan.

### Deacetylation

Chitin can be deacetylated partially or totally. Naturally occurring chitin is acetylated, with about 16% deacetylation. Chitosan refers to chitin that has been partially or fully deacetylated Chitosan is a polysaccharide formed primarily of repeating units of β (1-4) 2-amino-2-deoxy-D-glucose (or D-glucosamine). Further deacetylation of chitin can be achieved by processing of chitin. Deacetylation values can vary with chitin sources and with processing methods.

Since chitosan is made by deacetylation of chitin, the term degree of deacetylation (DAC) can be used to characterize chitosan. This value gives the proportion of monomeric units of which the acetylic groups that have been removed, indicating the proportion of free amino groups (reactive after dissolution in weak acid) on the polymer. DAC could vary from about 70% to about 100%, depending on the manufacturing method used. This parameter indicates the cationic charge of the molecule after dissolution in a weak acid. There are many methods of DAC measurements, such as UV and infrared spectroscopy, acid-base titration, nuclear magnetic resonance, dye absorption, and the like. Since there are no official standard methods, numbers tend to be different for different methods. In high value product, NMR can give a precise DAC number. However, titration or dye absorption can serve as a quick and convenient method and yield similar results as NMR.

Chitin deacetylation towards chitosan can be obtained by various methods. The most used method is that of alkaline treatment (Horowitz, S. T. et al., 1957). With this method, around 80% of deacetylation can be achieved without significant decrease of molecular weight. A more intense deacetylation cannot be obtained by this method without a simultaneous uncontrolled decrease of the degree of polymerization. A more promising method is deacetylation by a thermo-mechano-chemical treatment (Pelletier et al., 1990). This method allows a more careful control of the various characteristics of the final product (average degree of polymerization and of deacetylation). Finally, a third method (Domard and Rinaudo, 1983) allows obtainment of a totally deacetylated product.

In a certain deacetylation protocol, when chitin is heated in a basic solution, such as a strong solution of sodium hydroxide (such as about 40%) at high temperature (such as about 90° C.-120° C.), chitosan is formed by deacetylation. This treatment can remove acetylic grouping on the amine radicals to a product (chitosan) that could be dissolved. It is said that at least 65% of the acetylic groups should be removed on each monomeric chitin to obtain the ability of being put in solution. The degree of deacetylation will vary according to the treatment conditions, such as the duration, the temperature and the concentration of the basic solution.

In the preferred embodiments, the polychitosamine has a deacetylation higher than about 80%. Preferably, the polychitosamine has a deacetylation higher than about 89%. More preferably, the polychitosamine has a deacetylation higher than about 89, 90, 91, 92, 93, 94, 95, 96, 97, 98, 99, or 100%. In a polychitosamine that has been deacetylated about 100%, the advantage being the polychitosamine forms a relatively homogeneous composition.

According to the present invention, the polychitosamine has a molecular weight of about 30 kDa and is deacetylated at least 80%. In a preferred embodiment, the polychitosamine has a molecular weight of about 30 kDa and is deacetylated at least 93% and is sold under the trademark Libracol® in Canada.

### Fibrate

Fibrates are a class of amphipathic carboxylic acids. Their main actions are to decrease serum triglycerides and increase HDL. They act via activation of peroxysome proliferator-activated receptors (PPAR) a that are involved in the regulation of a number of genes involved in lipid metabolism. Fibrates are also effective at reducing the circulating concentration of small dense LDL. In an embodiment of the present invention, the fibrate is selected from the group consisting of ciprofibrate; gemfibrizil; benzafibrate; and fenofibrate. Preferably, fenofibrate is used in the combination therapy of the present invention.

### Synergism

Synergy or synergism, most often refers to the phenomenon of two or more discrete influences or agents acting in common to create an effect which is greater than the sum of the effects each is able to create independently.

Warnings of side effects from use of fibrate include renal impairment, severe hepatic impairment, hypoalbuminaemia, primary binary cirrhosis, gallbladder disease, and nephrotic syndrome. Some of these effects are noted when fibrate is taken in regular doses of at least about 1200 mg per day.

Accordingly, an advantage of using drug synergism is the ability to use a reduced amount of fibrate for the same result as in fibrate monotherapy, thus, resulting in fewer side effects.

### Prevention and Treatment of Conditions

The preferred embodiments can be used to prevent, give relief from, or ameliorate a disease condition having hyperlipidemia as an element of a disease, such as atherosclerosis or coronary heart disease, or to protect against or treat further high cholesterol plasma or blood levels with the compounds and/or compositions of the preferred embodiments. Hyperlipidemia is an elevation of lipids (fats) in the bloodstream. These lipids include cholesterol, cholesterol esters (compounds), phospholipids, triglycerides, and fatty acids. These lipids are transported in the blood as part of large molecules called lipoproteins.

Adverse events of hyperlipidemia include atherosclerosis and coronary heart disease. Atherosclerosis is a disease characterized by the deposition of lipids, including cholesterol, in the arterial vessel wall, resulting in a narrowing of the vessel passages and ultimately hardening the vascular system. The primary cause of coronary heart disease (CHD) is atherosclerosis. CHD occurs when the arteries that supply blood to the heart muscle (coronary arteries) become hardened and narrowed. As a result of CHD, there could be angina or heart attack. Over time, CHD can weaken your heart muscle and contribute to heart failure or arrhythmias.

Hypercholesterolemia is also linked to cardiovascular disease, Cardiovascular disease refers to diseases of the heart and diseases of the blood vessel system (arteries, capillaries, veins) within a person's entire body, such as the brain, legs, and lungs. Cardiovascular diseases include, but are not limited to, coronary heart disease, peripheral vascular disease, and stroke.

Accordingly, an embodiment of the present invention relates to the use of the combination therapy of polychitosamine and fibrate for lowering blood cholesterol in a mammal. Preferably, the mammal is a human.

Accordingly, another embodiment of the present invention relates to the use of the combination therapy of polychitosamine and fibrate in the prevention or treatment of hyperlipidemia and conditions associated with hyperlipidemia, such as hypercholesterolemia, atherosclerosis, coronary heart disease, and cardiovascular disease.

It should be understood that the combination therapy of polychitosamine and fibrate as mentioned hereinabove may be in the form of a pharmaceutical composition according to the present invention or any other composition comprising a combination of polychitosamine and fibrate. In the case of such composition, the nature and amount of polychitosamine and fibrate may be similar to those specified in the pharmaceutical composition of the present invention. A person skilled in the art could easily determine the exact amount based on the information provided in the instant application.

### Pharmaceutical Compositions

The compounds useful in the preferred embodiments can be presented with an acceptable carrier in the form of a pharmaceutical composition. The carrier is acceptable in the sense of being compatible with the other ingredients of the composition and is not deleterious to the recipient. The carrier can be a solid or a liquid, or both, and is preferably formulated with the compound as a unit-dose composition, for example, a capsule or tablet, which can contain from about 0.05% to about 95% by weight of the active compound. Examples of suitable carriers, diluents, and excipients include, but are not limited to, lactose, dextrose, sucrose, sorbitol, mannitol, starches, gum acacia, alginates, tragacanth gelatin, calcium silicate, cellulose, magnesium carbonate, or a phospholipid with which the polymer can form a micelle. Other pharmacologically active substances can also be present, including other compounds of the preferred embodiments. For example, more than one fibrate may be used together. The pharmaceutical compositions of the preferred embodiments can be prepared by any of the well-known techniques of pharmacy, comprising admixing the components.

In practicing the methods of the preferred embodiments, administration of the fibrate, in combination therapy, may be accomplished by oral route.

For oral administration, compounds used in the combination therapy can be in the form of, for example, but not limited to, a tablet, capsule, suspension, powders (e.g., for sprinkling on food), or liquid. Other embodiments include sustained-release capsules, enteric coated tablets, soft gel capsules, and other sustained-release technologies. Capsules, tablets, liquid, or powders, and the like can be prepared by conventional methods well-known in the art. The compounds are preferably made in the form of a dosage unit containing a specified amount of the compound Examples of dosage units are tablets or capsules.

Pharmaceutical compositions for use in the treatment methods of the preferred embodiments can be administered in oral form for compounds of the composition, or by parenteral, such as intravenous administration for the fibrate and oral administration of the polychitosamine. For convenience, oral administration of both therapeutic substances is preferred. Dosing for oral administration can be with a regimen calling for single daily dose, or for a single dose every other day, or for multiple, spaced doses throughout the day.

### Nutraceuticals

The compounds useful in the present invention can be incorporated in a functional food or nutraceutical. These compounds may be presented in the form of active agents such as cholesterol lowering agents. As such, these compounds may be useful in the manufacture of nutraceuticals and/or functional foods useful for preventing hyperlipidemia associated conditions.

In a preferred embodiment, the polychitosamine and fibrate compounds are incorporated in functional foods including but not limited to: beverages, including but not limited to sodas, water, sports/energy drinks, canned and bottled juices, fresh and refrigerated juices, frozen juices, yoghurt drinks, smoothies, teas and coffees; breads and grains, including but not limited to breakfast cereals, breads, baked goods, baking ingredients such as flour, frozen breads, dried breads and crackers, pastas; snack foods, including but not limited to nutrition bars, weight loss bars, energy/sports bars, candy bars, chips, gum; packaged and prepared foods, including but not limited to frozen foods such as pizzas and dinners, canned and dried soups, desserts including cookies; condiments, including but not limited to dressings, spreads, sauces; dairy and dairy alternatives, including but not limited to milk, cheese, butter, ice cream, yoghurt, margarine and soymilk.

### Dosages

A total daily dose of fibrate can generally be in the range of from about 50 mg/day to about 1200 mg/day in single or divided doses. Certain embodiments, with regard to appropriate dosage, are shown in Table 1.

TABLE I

| | DOSAGE PER ADMINISTRATION | | | | | | |
|---|---|---|---|---|---|---|---|
| | | | | Fenofibrate | Finofibrate | | |
| FREQUENCY OF | | | | (Lipidil | (Lipidil | | |
| ADMINISTRATION | HEP | Ciprofibrate | Fenofibrate | micro) | Supra) | Gemfibrozil | Benzafibrate |
| Once per day | 600 to 2,400 mg | 50 to 100 mg | 50 to 300mg | 70 to 300 mg | 50 to 200 mg | 800 to 1200 mg | 200 to 400 mg |
| (1 to 3 capsules) | | | | | | | |
| Per capsule | 200 to 800 mg | 16 to 100 mg | 16 to 300 mg | 23 to 300 mg | 16 to 200 mg | 200 to 800 mg | 60 to 400 mg |
| Twice per day | 400 to 1,200 mg | 25 to 50 mg | 25 to 150 mg | 35 to 150 mg | 25 to 100 mg | 300 to 600 mg | 100 to 200 mg |
| (1 to 2 capsules per) | | | | | | | |
| Per capsule | 200 to 800 mg | 12.5 to 50 mg | 12.5 to 150 mg | 18 to 150 mg | 12.5 to 100 mg | 150 to 800 mg | 50 to 200 mg |

In the case of pharmaceutically acceptable fibrate salts, the weights indicated above refer to the weight of the acid equivalent or the base equivalent of the therapeutic compound derived from the salt.

For a polychitosamine, a total daily dose of about 400 mg to about 4.8 grams per day and preferably between about 800 mg and about 2.4 grams per day may generally be appropriate. In an embodiment, polychitosamine is administered at about 400 mg per day. The polychitosamine is preferably taken three times a day. The polychitosamine is preferably taken with meals.

### Certain Dosages

As used herein, the term “total daily dose” refers to the amount of composition administered to an individual in one day.

The term “dose” refers to the amount of composition administered to an individual at one time.

The term “unit dose” refers to the amount of a composition pre-packaged by the manufacturer or pharmacist in standardized amounts. Thus, for example, the dose of ingredients in a single tablet or capsule would be considered a single “unit dose” whether one or more tablets or capsules are taken simultaneously.

In another embodiment, a polychitosamine is administered with a total daily dose of about 600 mg to about 2400 mg and a fibrate is administered at a total daily dose of about 50 mg to about 1200 mg. Preferably, the total daily dose is administered once per day in about 1 to about 3 unit doses, preferably in capsule form. Accordingly, each unit dose may contain about 200 mg to about 1200 mg of polychitosamine. Also, each unit dose may contain about 16 mg to about 600 mg of fibrate. In an embodiment, the total daily dose is administered in two unit doses administered once a day; the unit dose containing about 600 mg of polychitosamine and at least about 50 mg of fibrate. The dose is preferably administered with meals.

In another embodiment, the total daily dose is administered in two doses per day. In each dose, there is a polychitosamine in an amount of about 200 mg to about 1200 mg and a fibrate in an amount of about 50 mg to about 40 mg. Preferably, the total daily dose is administered in two doses per day with about 1 to about 2 unit doses per dose. Accordingly, each unit dose may contain about 200 mg to about 600 mg of polychitosamine. Also, each unit dose may contain about 25 mg to about 600 mg of fibrate. In an embodiment, the unit dose is administered in one capsule administered twice a day; the unit dose containing about 600 mg of polychitosamine and about 50 mg of fibrate. The dose is preferably administered with meals.

The dosage regimen to treat hyperlipidemia and hyperlipidemia-associated conditions, and reduce plasma cholesterol with the combination therapy and pharmaceutical compositions of the preferred embodiments is selected in accordance with a variety of factors. Those factors include, but are not limited to, the type, age, weight, sex, diet, and medical condition of the patient, the severity of the disease, the route of administration, pharmacological considerations, such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

Initial treatment of a patient suffering from a hyperlipidemic condition, such as, but not limited to, hypercholesterolemia, atherosclerosis, coronary heart disease, and cardiovascular disease, can begin with the dosages indicated above. Treatment should generally be continued as necessary over a period of several weeks to several months or years until the condition has been controlled or eliminated. Patients undergoing treatment with the compounds or compositions disclosed herein can be routinely monitored by, for example, measuring serum LDL and total cholesterol levels by any of the methods well known in the art, to determine the effectiveness of the combination therapy.

### Kits

The preferred embodiments also relate to kits for conveniently dispensing the combination therapy. These kits will preferably contain a plurality of daily doses of the fibrate and of the polychitosamine. The daily doses are preferably separate dosage forms of each medicament. Instructions are also provided for patient and/or dispensing physician or pharmacist. The kits may contain supplies for a given time duration of treatment such as one month.

Blister package with 7 days worth of treatment, each day indicated (Monday, Tuesday, Wednesday, Thursday, Friday, Saturday, Sunday). Each day has two capsules or tablets, one indicated as “breakfast”, the other indicated as “supper”. There are a total of 14 capsules per blister pack (2 rows and 7 columns) and four blisters in a box—enough for a four week (28 days) supply. Alternatively, you could have one single row for once daily dosing (dinner only), or three rows for three times per day dosing (breakfast, lunch, dinner). According to this embodiment, the fibrate and the polychitosamine are contained in the same capsule or tablet.

According to another embodiment, the fibrate and the polychitosamine are in separate tablets or capsules, one dose per day of each, for each of seven days. This would represent 2 rows and 7 columns per blister, with 4 blisters per box.

Those skilled in the art will know, or be able to ascertain, using no more than routine experimentation, many equivalents to the specific embodiments of the invention described herein. These and all other equivalents are intended to be encompassed by the following claims.

Although the present invention has been explained hereinabove by way of a preferred embodiment thereof, it should be pointed out that any modifications to this preferred embodiment within the scope of the appended claims is not deemed to alter or change the nature and scope of the present invention.

### EXAMPLE 1

1.0 SUMMARY OF THE TRIAL

| Name of | HEP-40 ™ (40 kDa, 93% deacetylated, | |
|---|---|---|
| product | polychitosamine) | |
| studied | | |
| Trial title | A 12 week, double blind, placebo controlled, | |
| | randomized pilot study evaluating the | |
| | effectiveness of HEP-40 ™ co-administered with a | |
| | fibrate in patients with persistent | |
| | hypercholesterolemia while on fibrate | |
| | monotherapy. | |
| Estimated trial | Treatment: 12 weeks. | |
| start & | | |
| duration | | |
| Hypotheses | Primary: | |
| | In patients with persistent hypercholesterolemia | |
| | defined as above target LDL-C while on fibrate | |
| | monotherapy, a 12 week course of 800 mg bid of | |
| | HEP-40 ™ co-administered with the current fibrate | |
| | will produce a significantly higher reduction in | |
| | circulating serum LDL-C when compared to | |
| | placebo. | |
| | Secondary: | |
| | i. | In patients with persistent |
| | | hypercholesterolemia defined as above |
| | | target LDL-C while on fibrate |
| | | monotherapy, a 12 week course of |
| | | 800 mg bid of HEP-40 ™ co-administered |
| | | with the current fibrate will produce |
| | | significantly higher reductions in the |
| | | following lipid parameters when |
| | | compared to placebo: |
| | | Triglycerides |
| | | Total cholesterol |
| | | C-Reactive Protein |
| | ii. | In patients with persistent |
| | | hypercholesterolemia defined as above |
| | | target LDL-C while on fibrate |
| | | monotherapy, a 12 week course of |
| | | 800 mg bid of HEP-40 ™ co-administered |
| | | with the current fibrate will be well |
| | | tolerated and safe. |
| Primary | In patients with persistent hypercholesterolemia | |
| objective | defined as above recommended target LDL-C | |
| | while on fibrate monotherapy: | |
| | To compare the effectiveness of a 12 week course | |
| | of 800 mg bid of HEP-40 ™ versus placebo co- | |
| | administered with the current fibrate in reducing | |
| | circulating serum LDL-C. | |
| Secondary | In patients with persistent hypercholesterolemia | |
| objectives | defined as above recommended target LDL-C | |
| | while on fibrate monotherapy: | |
| | i. | To compare the effectiveness of 800 mg |
| | | bid of HEP-40 ™ versus placebo co- |
| | | administered with the current fibrate at |
| | | an unaltered dose in reducing the |
| | | following lipid parameters: |
| | | Triglycerides. |
| | | Total cholesterol |
| | | C-Reactive Protein |
| | ii. | To assess the safety and tolerability of a |
| | | 12 week course of 800 mg bid of HEP- |
| | | 40 ™ co-administered with the current |
| | | fibrate. |

| | |
|---|---|
| Clinical phase | Phase IV Pilot Study |
| Trial design | Double blind, placebo controlled, randomized trial |
| | Patients with persistent hyperlipidemia, defined |
| | as LDL-C levels above recommended target |
| | while on fibrate monotherapy will be enrolled. |
| | Patients must be on fibrate therapy for more than |
| | 4 weeks but less than 6 months. |
| | Patients will be randomized in a 1:1 ratio to be |
| | treated with: |
| | HEP-40 at 800 mg bid co-administered with |
| | their current fibrate for 12 weeks (HEP-40) |
| | placebo bid co-administered with their current |
| | fibrate for 12 weeks (PLACEBO) |
| Number of | At least 20 patients will be enrolled and |
| patients to be | randomized. |
| included | At least 14 evaluable patients are required. |
| Formulation, | Fibrate treatment will continue as per prior to |
| route of | study entry. Study drug (HEP-40 or placebo) will |
| administration | be administered orally during meals. Placebo and |
| & dosage | HEP- 40 will be similar in formulation, |
| | appearance and taste. |
| Treatment | 12 weeks of HEP-40 ™ or Placebo co- |
| duration | administered with the current fibrate dose. |

1. A pharmaceutical dosage form comprising in combination:
a) an amount of fibrate;
b) an amount of polychitosamine having a molecular weight ranging from about 30 kDa to about 50 kDa; and optionally
c) a pharmaceutically acceptable carrier.

2. (canceled)

3. The dosage form according to claim 1, wherein the polychitosamine has a degree of deacetylation ranging from about 80% to about 100%.

4. The dosage form according to claim 3, wherein the polychitosamine has a molecular weight of about 30 kDa and a degree of deacetylation of about 89%.

5. The dosage form according to claim 4, wherein the polychitosamine is Libracol®.

6. The dosage form according to claim 3, wherein the polychitosamine has a molecular weight of about 40 kDa and a degree of deacetylation of at least about 80%.

7. The dosage form according to claim 1, wherein the fibrate is fenofibrate.

8. The dosage form according to claim 1, wherein it comprises at least about 25 mg of fibrate.

9. The dosage form according to claim 1, wherein it comprises at least about 400 mg of polychitosamine.

10-22. (canceled)

23. A method for preventing or treating hyperlipidemia in a mammal comprising administering to said mammal an effective amount of the dosage form of claim 1.

24. The method according to claim 23, wherein the amount of fibrate administered to the mammal is at least about 50 mg per day.

25. The method according to claim 23, wherein the amount of polychitosamine administered to the mammal is at least about 600 mg per day.

26. The method according to claim 23, wherein the mammal is a human.

27. A method for lowering blood cholesterol in a mammal, wherein said method comprises the step of administering to said mammal an effective amount of the dosage form of claim 1.

28. The method according to claim 27 wherein the amount of fibrate administered to the mammal is at least about 50 mg per day.

29. The method according to claim 27 wherein the amount of polychitosamine administered to the mammal is at least about 600 mg per day.

30. The method according to claim 27 wherein the mammal is a human.

31. A functional food comprising an effective amount of polychitosamine having a molecular weight ranging from about 30 kDa to about 50 kDa and an effective amount of fibrate.

32. The functional food according to claim 31 selected from the group consisting of beverages, bread, grain, snack foods, prepared and packaged foods, condiments and dairy and dairy alternatives.

33. (canceled)

34. The functional food according to claim 31 wherein the polychitosamine has a degree of deacetylation ranging from about 80% to 100%.

35. The functional food according to claim 31 wherein the polychitosamine has a molecular weight of about 30 kDa and a degree of deacetylation of about 89%.

36. The functional food according to claim 35 wherein the polychitosamine is Libracol®.

37. The functional food according to claim 31 wherein the fibrate is fenofibrate.

38. The functional food according to claim 31 wherein it comprises at least about 25 mg of fibrate.

39. The functional food according to claim 38 wherein it comprises at least about 400 mg of polychitosamine.

40. A kit for the prophylaxis or treatment of hyperlipidemia in a mammal, the kit comprising a plurality of daily doses of dosage forms of a fibrate and a polychitosamine having a molecular weight ranging from about 30 kDa to about 50 kDa together with a treatment regimen instructions.

41. The kit according to claim 40, wherein the daily dose of fibrate is at least about 50 mg per day.

42. The kit according to claim 41, wherein the daily dose of polychitosamine is at least about 600 mg per day.

43. The dosage form according to claim 1, wherein the dosage form comprises a pharmaceutically acceptable carrier.

44. The dosage form according to claim 3, wherein it comprises at least about 25 mg of fibrate.

45. The dosage form according to claim 44, wherein it comprises at least about 400 mg of polychitosamine.

46. The dosage form according to claim 45, wherein the fibrate is fenofibrate.

47. The dosage form according to claim 46, wherein the polychitosamine has a molecular weight of about 30 kDa and a degree of deacetylation of about 89%.

48. The dosage form according to claim 47, wherein the polychitosamine is Libracol®.
